(19) United States
(12) Patent Application Publication
(10) Pub. No.: US 2013/0216545 A1
(43) Pub. Date: Aug. 22, 2013

(54) Early Diagnosis and Novel Treatment of Cancer

(71) Applicant: Claresa Levetan, Bryn Mawr, PA (US)

(72) Inventor: Claresa Levetan, Bryn Mawr, PA (US)

(21) Appl. No.: 13/400,092

(22) Filed: Feb. 19, 2012

(51) Int. Cl. A61K 39/395; A61K 31/7088; A61P 37/04; C07K 16/18; A61K 39/00; C07H 21/02; A61P 35/00

(52) U.S. Cl. 424/139.1; 536/24.5; 514/44 A; 530/389.1; 424/184.1; 424/130.1; 424/185.1

(57) ABSTRACT

The present invention relates to novel therapies for treatment and new biomarkers for earlier detection of pancreatic and other cancers. In particular, the present invention identifies Reg1α and Reg3α, and other members of the Reg protein family, as signaling proteins for a receptor on the surface of human cancer cells, Megi, and the downstream pathways activated through this receptor in pancreatic and other tumor cells. These signaling proteins may be targeted by means known in the art to disrupt the downstream signaling pathway that catalyzes tumorigenesis, by the usage of antibodies, antisense, RNA interference, small molecule inhibitors and vaccines.

### SEQUENCE LISTING

The instant application contains a Sequence Listing which has been submitted in ASCII format via EFS-Web and is hereby incorporated by reference in its entirety. Said ASCII copy, created on Feb. 2, 2012, is named LEVE0001.txt and is 18,615 bytes in size.

### FIELD OF THE INVENTION

The present invention relates to novel therapies for treatment and new biomarkers for earlier detection of pancreatic and other cancers, through the identification of signaling proteins, their receptor on the surface of human cancer cells and the downstream pathways activated through this receptor in pancreatic and other tumor cells. In particular, the present invention identifies novel ligands and a receptor, when targeted by means known in the art to disrupt and block this receptor, limiting its downstream signaling pathway that catalyzes tumorigenesis, by the usage of antibodies, antisense, RNA interference, small molecule inhibitors, including a therapeutic cancer vaccine derived from unique binding regions of the signaling proteins and their receptor, which may also be used in high risk patients for the prevention of cancer. This invention also includes new plasma biomarkers for earlier identification of tumors, also for assessing tumor activity and efficacy of therapy and new tissue biomarkers to evaluate tumors most responsive to therapies herein.

### BACKGROUND OF THE INVENTION

Pancreatic cancer is a disease with a high rate of mortality with a one-year survival of only 25% and a 5-year survival rate of only 5%. There are no serum biomarkers for pancreatic cancer, nor are there specific therapies uniquely designed for pancreatic cancer based on identified overexpressed genes or receptors on pancreatic tumor cells. Because there have also been no adequate screening tools, cancer of the pancreas is almost always diagnosed at advanced stages, when no successful therapies are available. Patients present with non-specific clinical findings, with the most alerting being painless jaundice.

By the time the diagnosis is made by surgical removal of the cancer after a tumor is identified by a radiological test, most patients have tumors that have invaded the lymphatics and blood vessels. A typical feature of pancreatic cancer is that early on, it spreads both locally and distantly to other organs. In clinical practice, the most common form of pancreatic cancer, also known as pancreatic ductal adenocarcinoma, accounts for 95% of malignant tumors arising from the pancreas.

There is a need to identify new target, molecules that are involved in tumorigenesis for the treatment and prevention of cancer and identify plasma tumor markers that help in the earlier identification of cancer among populations at risk.

It is recognized that many types of human cancers, including tumors of the gastrointestinal tract, pancreatic, colon, rectal, gastric, and esophagus may result from the uncontrolled expression of normally occurring human genes. With normal regulatory feedback, genes turn on and off as necessary for the body to function. For example, some genes turn on almost exclusively during embryogenesis when the fetus is populating an organ for the first time with new cells. These genes are only upregulated again after fetal development when there is an acute organ injury, when there is a need for regeneration of that organ, but at other times, there is unwarranted, dysregulated cell growth due to constitutive gene expression as in the case of many cancers.

It is also known from the Human Genome Project that the genes that are responsible for making cells of the different organs and populating organs for the first time (such as the brain, heart, kidney and pancreas) reach their peak expression during the first year of life. Only recently, have certain cancers, including pancreatic cancer, as demonstrated in this invention, been shown to represent the abnormal expression of genes that are not typically seen expressed in adults, and these gene proteins are directly involved in tumorigenesis.

The regenerating gene family of proteins (Reg) is composed of various acute phase reactants, lectins, antiapoptotic factors, and growth factors affecting pancreatic islet cells, neural cells, and epithelial cells within the digestive system. To date, 17 Reg family genes have been identified. Identified members of the human REG gene family (REG) include Regenerating islet-derived 1 alpha (REG 1α or REG 1a), REG Iβ, REG IIIα, REG IIIβ and REG IV.

The human. REG IV gene family and the Reg IV protein family have been identified as potential targets for therapeutic intervention for the treatment of colon cancer. See, e.g., U.S. Pat. No. 7,510,708; Yu-Wei Zhang et al., World J Gastroenterol (2003), 9(12): 2635-2641; Kumar S. Bishnupuri, et al., Gastroenterology (2010), 138(2): 606-610. Although other Reg proteins has been identified as being present in tumor states, for example, the regenerating islet-derived 1 gene (REG 1) and expression of the Reg1 protein in the proliferation of gastrointestinal tumors, including colon, hepatocellular and gastric carcinomas, but the role of Reg1a, Reg3a and other Reg gene proteins has been considered that of an inflammatory marker rather than an active participant in the tumorigenesis pathway. Expression of the Reg 1α and Reg1β proteins has been observed in colon tumors. H. Rechreche et al. (1999), 81: 688-694. Mutations of REG that inhibit secretion of Reg 1 have also been associated with gastric enterochromaffinlike (ECL cell) carcinoids, suggesting that Reg 1 may function as an autocrine or paracrine tumor suppressor. Yu-Wei Zhang et al., World J. Gastroenterol (2003), 9(12): 2635-2641.

The REG1α gene encodes a protein secreted by the exocrine pancreas, which has been associated with islet cell regeneration. Terazono K., et al., J Biol Chem (1988), 263: 2111-21114. Regia expression has also been shown to be expressed in breast, lung, stomach, bile duct, colon and esophageal cancer. Sasaki K., et al., Annals of Surgical Oncology (2008), 15(11): 3244-3251; Astrosini C., et al., Int. J. Cancer (2008), 123: 409-413.

Regenerating islet-derived 3 alpha (REG 3α or REG 3a) is a gene that encodes a pancreatic secretory protein (Reg3α or Reg3a) that may be involved in cell proliferation or differentiation. The enhanced expression of Reg3α is observed during pancreatic inflammation and liver carcinogenesis. Reg3α was first isolated as a pancreatic secretory protein implicated in pancreatic regeneration, and then identified in human hepatocellular carcinomas (HCC). In the exocrine pancreas, Reg3α protein is associated with pancreatic acinar cell protection from oxidative stress and tumor necrosis factor-alpha (TNFα) induced pancreatic stress. It has also been reported to behave as a mitogenic factor towards hepatocytes, and an antiapoptotic molecule against TNFα mediated hepatocyte apoptosis. Cavard C., et al. Oncogene (2006), 25: 599-608.

REG 3α and REG 1α belong to the C-lectin family and are tandomly clustered on 2p12. They share structural and some functional properties and encode proteins that are members of the Reg family with substantial sequence homology. Their products are secretory proteins of the C-type lectin superfamily that are involved in liver and pancreatic regeneration and proliferation. Consequently, they are candidates for involvement in liver tumorigenesis. They are strongly induced in a b-catenin activated liver adenoma and carcinoma, suggesting a common regulation of these genes by the b-catenin pathway. Cavard C., et al. Oncogene (2006), 25: 599-608.

Although it has been suggested that the REG family of genes are present in certain cancers, the role they play has remained unclear with most of the literature considering Regla and Reg3α as markers for inflammation. There has been limited data on Reg1α and Reg3α playing a role in the progression of pancreatic cancer. It is known that regenerating islet-derived 1 alpha (Reg1α or Reg1a) and Reg3α are proteins not normally expressed in healthy pancreas and other healthy organs. Although Reg1α and Reg3α have previously been described as being present in liver and colon tumors and also in acute and chronic pancreatitis, the roles for these two proteins have, until recently, been considered as markers produced by the tumor or a potential inflammatory marker. Further, it has not been suggested that Reg1α or Reg3α play a role in triggering a signal cascade that leads to increased tumorigenesis.

The human hereditary Multiple Exostoses Gene Isolog (MEGI or EXTL3) was named for its similarities to the Exostoses family of genes by homology screening, but it was specifically noted that the Megi protein (Megi) is not derived from the Exostoses (EXT and EXTL) genes. Rather, the Megi protein was categorized as a member of the Exostosin family because it demonstrates a 52% homology to the 262 amino acid C-terminal of the Exostosin-like 2 protein and a 40% homology with the 242 amino acid C-terminal of the Exostosin-like 1 protein, yet there is no homology of Megi to the N-terminal regions of Exostosin-1 or 2. See, e.g., Kobayashi S. et al., Anat. Embryol. 207:11-15, 2003. Megi was initially isolated and described by Van Hul and colleagues in 1998 as a 919 amino acid protein (SEQ ID NO: 1). Megi contains a 23 amino acid unique N-terminal region containing a transmembrane domain (residues 28-51) (SEQ ID NO: 2) and a short intracellular region at the N terminus. Megi is located on chromosome 8p21. See, e.g., Saito T. et al, Biochem Biophys Res Commun. 1988, 242(1):61-66, Van Hul W et al., Genomics. 1′998; 47(2):230-7. Megi is also known as the BOTV, BOTY, DKFZp686C2342, exostoses (multiple)-like 3, Exostosin-like 3, EXT-related protein 1, EXTL1L, HHMEGI, EXTR1, EXTL3 Glucuronyl-galactosyl-proteoglycan-4-alpha-N-acetylglucosaminyltransferase, KIAA0519, Multiple exostosis-like protein 3, REG, HHMEGI, REGR and RPR.

The N-terminal region (residues 1-656) of Megi has no homology to any other members of this family of genes. The 1.6-kbp cDNA, which was initially isolated in the screening of the rat islet cDNA expression library as a Reg-binding protein, contained the N-terminal region alone (amino acid residues 1-332). (Kobayashi S. et al., Anat. Embryol. 207:11-15, 2003). Because no other members of the EXT family bind to Reg proteins, the Reg binding domain likely exists in the N-terminal region of Megi.

While it has been recognized that Megi may act as a rodent Reg family receptor for Regia, to date, it has not been suggested that Megi is present on human cancer cells or that members of the Reg family directly or indirectly interact with a Megi receptor on tumor cells as a signaling mechanism leading to tumor growth and production. Further, no data has previously confirmed the role of Megi as a receptor for Reg or its direct involvement in the development of tumorigenesis.

The overexpression of MEGI enhances the expression of nuclear factor-kβ (NF-kβ) activity stimulated by TNFα. K. Mizuno et al., Cellular Signaling (2001) 13:125-130. It has also been theorized that Megi (i.e., Ext13) is a receptor for Reg1 in rats. See U.S. Pat. No. 7,510,708. However, to date it has not been suggested that Reg1α and Reg3α, and potentially other members of the Reg family, bind to Megi to cause a downstream reaction resulting in the production of NF-kβ and TNFα, and lead to tumor production and growth.

There have been two previous reports of the immunohistochemical presence of Reg3α as an inflammatory marker in pancreatic cancer in up to 87% of human pancreatic cancer compared to the absence of staining in healthy pancreata (Xie M-J, Dig Dis Sciences, (2003), 48(3):459-64; Motoo Y, Dig Dis Sci, (1999), 44(6):1142-7). Despite the documentation of Reg1α and Reg3α in human tumors, previous reports hypothesized that Reg1α and Reg3α are increased secondary to inflammatory cytokines, such as TNFα, specifically reported that Reg1α and Reg3α are not signaling peptides in the pathway for tumor expansion and growth, as reported in this invention.

The present invention demonstrates that Megi can readily be identified on the surface of human pancreatic tumors and that Megi action is accelerated by the interaction with Reg1α and Reg3α (and other members of the Reg family). Findings demonstrate that Megi plays a key regulatory role in tumor growth and expansion, including pancreatic adenocarcinoma. Further, the present invention identifies the binding region within Reg1α and Reg3α, which is common to other members of the Reg family, and a binding domain on Megi, which are targets for the treatment and prevention of tumor growth.

The present invention further demonstrates that in human cancers, including the most common form of human adenocarcinoma of the pancreas, Megi is a unique rate-limiting receptor on the surface of tumor cells and is a pivotal receptor site for cancer cell growth, acceleration and turnover with data suggesting that blockade of Megi, limits Reg1α and Reg3α action in human pancreatic adenocarcinoma. The present invention also demonstrates that the overexpression of Reg1α or Reg3α enhances tumor cell rate of growth.

### BRIEF SUMMARY OF THE INVENTION

In a first embodiment of the invention, an antisense construct is delivered to a tumor cell to inhibit the uniquely identified Megi receptor on human tumor cells and/or the interaction of Reg1α and Reg3α with Megi on human cancer cells. In one embodiment, the construct preferably comprises at least 7 nucleotides, preferably at least 10 nucleotides, preferably at least 13 nucleotides, or preferably at least 15 nucleotides of the complement of the human, mouse or rat MEGI gene, or the rat, mouse, or human REG gene family cDNA selected from the group consisting of REG 1α, REG 1b, REG 3α and REG IV. As a result of the delivery, the tumor cell expression is limited with diminished tumor cell growth and expression.

In a second embodiment of the invention, an RNA interference construct is delivered to a tumor cell. The construct preferably comprises at least 7 nucleotides, preferably at least 10 nucleotides, preferably at least 13 nucleotides, or preferably at least 15 nucleotides of the human, rat or mouse MEGI, rat, mouse, or human REG gene family cDNA selected from the group consisting of REG 1α, REG 1b, REG 3α and REG IV. As a result of the delivery, the tumor cell expression is limited with diminished tumor cell growth and expression.

According to a third embodiment of the invention a method is provided for causing cleavage of MEGI or REG gene mRNA produced by a tumor cell. A siRNA is delivered to the tumor cell. The siRNA preferably comprises at least 7 bp duplexes, preferably at least 10 bp duplexes, preferably at least 13 bp duplexes, or preferably at least 15 bp duplexes of human, rat or mouse MEGI, or rat, mouse, or human REG gene family RNA selected from the group consisting of REG 1α, REG 1b, REG 3 α and REG IV. The siRNA has 2 nt 3′ overhangs.

According to a fourth embodiment of the invention a method is provided for identifying a test substance that inhibits binding of a Reg protein to the Megi protein. A rat, mouse, or human Megi protein is contacted with a rat, mouse, or human Reg protein in the presence and in the absence of a test substance. The Reg protein is selected from the group consisting of Reg1α, Reg1β, Reg3α and RegIV. Binding of the Reg protein to the Megi protein is determined in the presence and in the absence of a test substance. A test substance which inhibits binding of the Reg protein to the Megi protein is identified.

According to a fifth embodiment of the invention an inhibitor is delivered to a tumor cell. The inhibitor inhibits the binding of a rat, mouse, or human Megi protein to a rat; mouse; or human Reg protein selected from the group consisting of Reg1α, Reg1β, Reg3α and RegIV. Resistance to apoptosis by the tumor cell is thereby reduced.

According to a sixth embodiment of the invention an antibody is delivered to a tumor cell. The antibody specifically binds to a rat, mouse, or human Reg family protein selected from the group consisting of Reg1α, Reg1β, Reg3α, RegIV, and Megi. Binding of Megi to a Reg protein in the tumor cell is thus inhibited.

In a seventh embodiment of the present invention, a therapeutic antigen/adjuvant cancer vaccines (for example, pancreatic cancer) are derived from specified regions of the Megi protein and other vaccines are derived from specified regions of Reg proteins, may be used to prevent tumor development based on targeting Megi, Reg1α, Reg1β, Reg3α or RegIV.

In an eighth embodiment of the present invention, methods are provided for screening, earlier detection of tumors and monitoring plasma and tissue for the presence and quantitation of Megi, Reg1α, Reg1β, Reg3α and RegIV as biomarkers to 1) evaluate plasma among patients at risk for or may have occult adenocarcinoma of the pancreas or other tumors expressing Megi, Reg1α, Reg1β, Reg3α or RegIV in order to make an earlier diagnosis of cancer 2) evaluate patient tumor burden among patients diagnosed with pancreatic cancer and other cancers expressing Megi, Reg1α, Reg1β, Reg3α or RegIV 3) evaluate tumors for Megi, Reg1α, Reg1β, Reg3α or RegIV to better evaluate therapeutic options for patients who have tumors expressing Megi, Reg 1α, Reg1β, Reg3α or RegIV, and 4) monitor plasma for the efficacy of treatment among patients with tumors expressing Megi, Reg1α, Reg1β, Reg3α and RegIV.

Development of tumor biomarkers for Megi, Reg1α, Reg1β, Reg3α and RegIV may include but is not limited to qualitative testing including immunohistochemical studies (for example, monoclonal anti-Megi antibody on human cancer cells, anti-human Reg1α antibody on human cancer cells, and anti-human Reg3α antibody on human cancer cells) and a tissue and plasma assay utilizing reverse transcription polymerase chain reaction the exponential amplification via reverse transcription polymerase chain reaction for a highly sensitive evaluation of Megi, Reg3α and Reg1α as a tool for both earlier identification and monitoring treatment status.

In a ninth aspect of the invention, novel tools are provided for serum screening for Megi, Reg1α, Reg1β, Reg3α and RegIV for populations at risk, including, for example, those patients with diabetes who have a marked increased risk for both pancreatic and breast cancer, which includes patients with both type 1 and type 2 diabetes who carry a two-fold or greater risk for pancreatic cancer and women with diabetes whose risk is 37% higher than nondiabetic individuals.

In another aspect of the present invention, inhibitors, antisense constructs and other molecules can be designed to target Megi, Regia and Reg3α (and other Reg molecules such as Reg1β and RegIV) to treat cancer and reduce the size of tumors. In another embodiment of the invention, inhibitors, antisense constructs and other molecules can be designed to target the amino acid regions within Megi to include, but not limited to SEQ ID NOS: 3 and 4 and sequences within amino acids 1-332 of SEQ ID NO: 1, which contains a unique N-terminal region of Megi that is not contained in any other member of the Exostoses family, and no other Exostoses family member have been shown in rodent models to bind Reg proteins.

In another aspect of the present invention a vaccine is designed both from Megi and the Reg proteins to treat cancer and reduce the size of tumors. In another embodiment of the invention, therapeutic vaccines are designed to target the amino acid regions within Megi to include, but not limited to SEQ ID NOS: 3 and 4 and sequences within amino acids 1-332 of SEQ ID NO: 1, which contains a unique N-terminal region of Megi that is not contained in any other member of the Exostoses family, and no other Exostoses family member have been shown in rodent models to bind Reg proteins. Additionally, therapeutic vaccines are designed from Reg1α, Reg3α Reg1β and RegIV (for example, SEQ (ID NOS: 5 through 8), and from homologous peptide regions (for example, SEQ ID NO: 9) contained in Reg1α, Reg3α, Reg1β and RegIV

The present invention also includes markers for novel screening and monitoring methods for cancer, for example, pancreatic cancer.

In a further embodiment of the invention, Reg1α, Reg3α (and other Reg molecules such as Reg1β and RegIV) and Megi are serum markers utilized as screening tools for early detection of pancreatic and other cancers in high risk populations, such as diabetes and pancreatitis.

Other embodiments of the present invention provide pharmaceutical formulations and unit dose forms of inhibitors and competitors of Megi and its downstream pathway.

Yet other embodiments of the present invention include methods of treating a pathology associated with pancreatic cancer and other cancers expressing MEGI, REG, for example REG 1α or REG 3α, and among those tumors in which such genes are candidates, in a subject in need of such treatment.

In another embodiment of the present invention, methods of diagnosing and treating pathology associated with cancer in a subject in need of such treatment are provided.

### BRIEF DESCRIPTION OF THE DRAWINGS

FIG. 1 is a schematic drawing showing the findings of the present invention that the Reg gene proteins, Reg1α and Reg3α, act through the Megi receptor on pancreatic tumor cells, stimulating more rapid translocation of Megi from the cytoplasmic membrane to the nucleus with downstream activation of tumor cell growth and enhancement of Tumor Necrosis Factor-Alpha (TNFα) and NF Kappa Beta (NF-1β) in adenocarcinoma.

FIG. 2 is an illustration showing the three dimensional structures of Reg1α and Reg3α based on their primary amino acid sequences and folded by SwissProt folding algorithms. FIG. 2a shows the three dimensional structure for Reg1α, and FIG. 2b shows the three dimensional structure for Reg3α. The binding region of Reg1α and the homologous region on Reg3α that bind to the Megi receptor are circled.

FIG. 3 is an alignment of the 166 amino acid sequence for Reg1α (SEQ ID NO:5), the 166 amino acid sequence for Reg1β (SEQ ID NO: 6), the 175 amino acid sequence for Reg3α (SEQ ID NO:7), and the 158 amino acid sequence for RegIV (SEQ ID NO:8) showing a common Reg peptide sequence (SEQ ID NO: 9) that was synthesized (SEQ ID NO: 10) for evaluating the interaction between Reg and Megi in adenocarcinoma (boxed sequence).

FIG. 4 shows the 919 amino acid sequence for Megi (SEQ ID NO: 1) with the 23 amino acid membrane spanning domain (SEQ ID NO: 2) boxed.

FIG. 5 is an illustration showing the three dimensional structure of Megi based on its primary amino acid sequences and folded by SwissProt folding algorithms. FIG. 5a shows the three dimensional structure from the front of Megi, FIG. 5b shows the three dimensional structure from the top of Megi, and FIG. 5c shows the three dimensional structure from the side of Megi.

FIG. 6 shows immunofluorescent staining of Megi on the cell surface of adenocarcinoma cells. In utilizing Cy3 immunofluorescent staining of Megi in human pancreatic adenocarcinoma cells in standard medium, there is immunofluorescent staining of Megi, which is well-defined at the cell borders indicating surface expression of Megi on the cytoplasmic membrane of cells. (FIG. 6a). FIG. 6b demonstrates the difference in Megi staining when cells are exposed to Reg peptide (SEQ ID 10).

FIG. 7 shows Western blot analysis of Megi levels with the presence of Megi in the nucleus within 30 minutes with and without Reg peptide is present. FIG. 7 shows the presence of Megi in the cytoplasm at 30 minutes and 6 hours alone (FIGS. 7a, 7b, 7e, 70 and in the presence of Reg peptide (FIGS. 7c, 7d, 7g, 7h) in serum free medium (SFM). FIG. 7 shows that the addition of Reg in serum free culture medium (SFM) enhanced Megi nuclear translocation, demonstrated by higher nuclear levels of this protein at 30 minutes.

FIG. 8 shows the immunofluorescent analyses of Megi translocation from cell membrane to cancer cell nucleus. Immunofluorescent analyses of Megi are shown in the upper panels (FIG. 8a, FIG. 8b and FIG. 8c) indicated by the Cy3 immunofluorescent staining of Megi. In the lower panels (FIG. 8d, FIG. 8e and FIG. 80 the Cy3 immunostaining of Megi has been overlaid with 4′,6-diamidino-2-phenylindole dihydrochloride (DAPI) staining the nuclei in blue (indicated by the light grey contrast in FIGS. 8d, 8e and 8f). Cells were grown in standard growth medium as a control and compared to cells grown serum-free medium (SFM) in the presence or absence of 150 μM of Reg peptide. The arrows in FIG. 8a and FIG. 8d demonstrate examples of the surface expression of Megi grown in standard growth medium. The well-defined cell borders indicate surface expression of Megi on the plasma membrane. The arrows in FIG. 8a and FIG. 8d delineate the cell borders, whereas the nuclei are shown in light grey contrast in FIG. 8d.

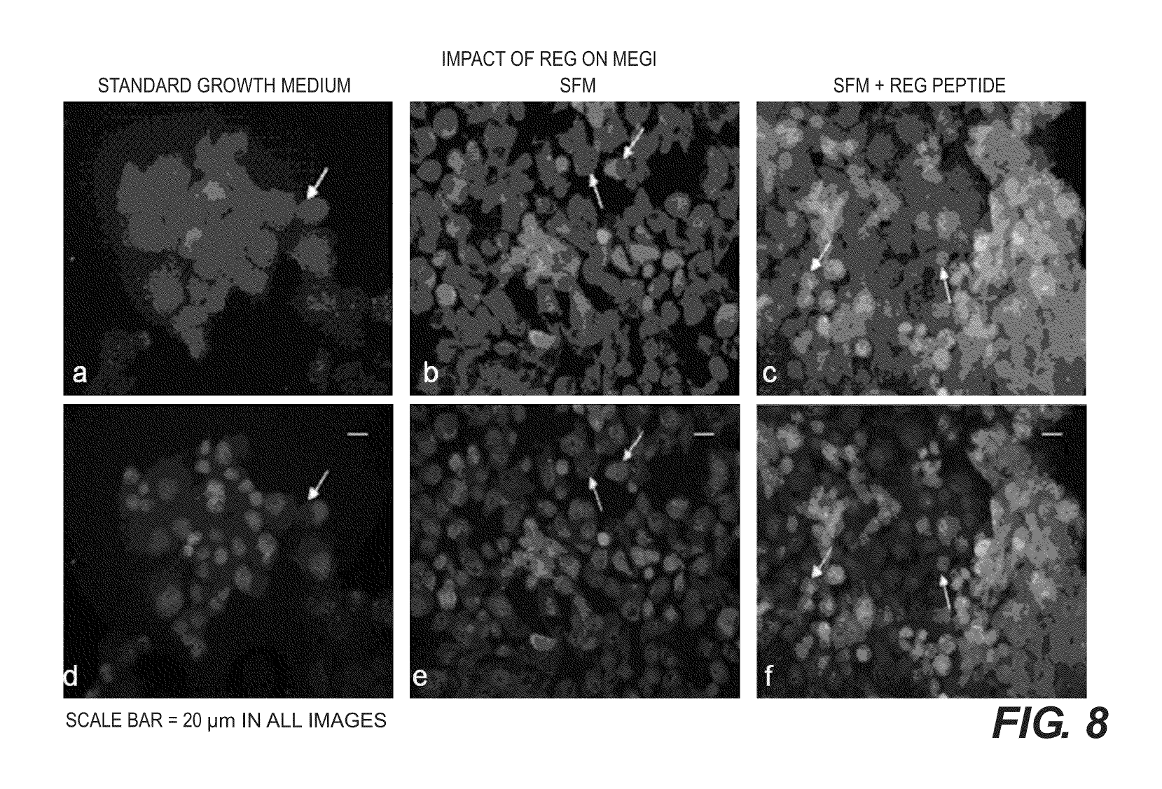

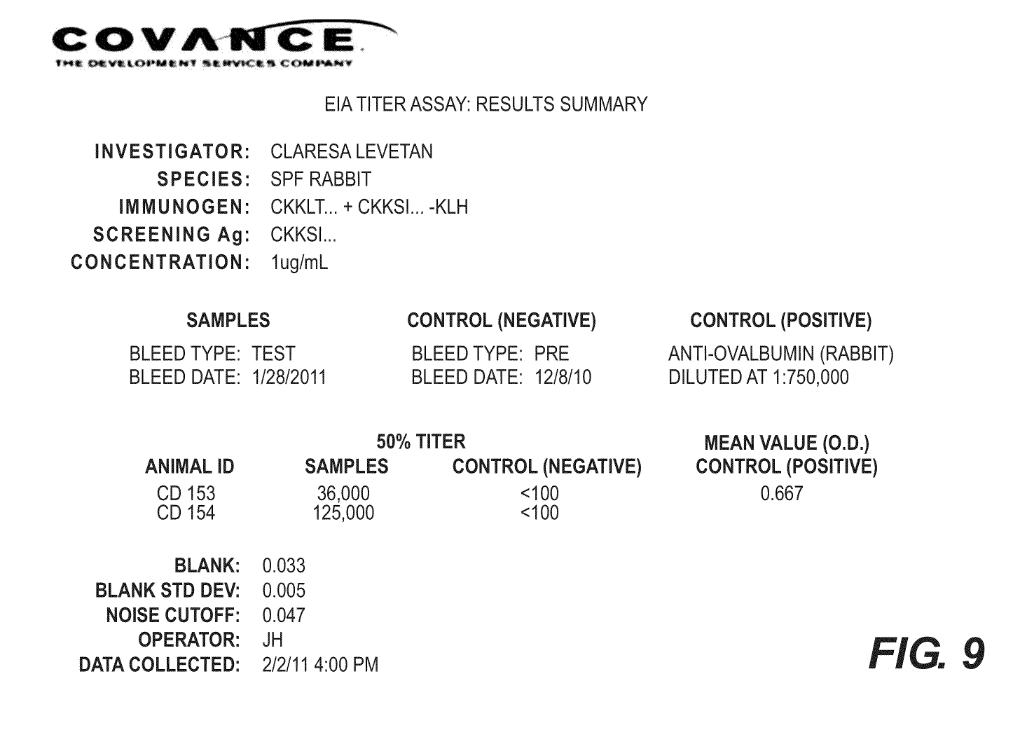

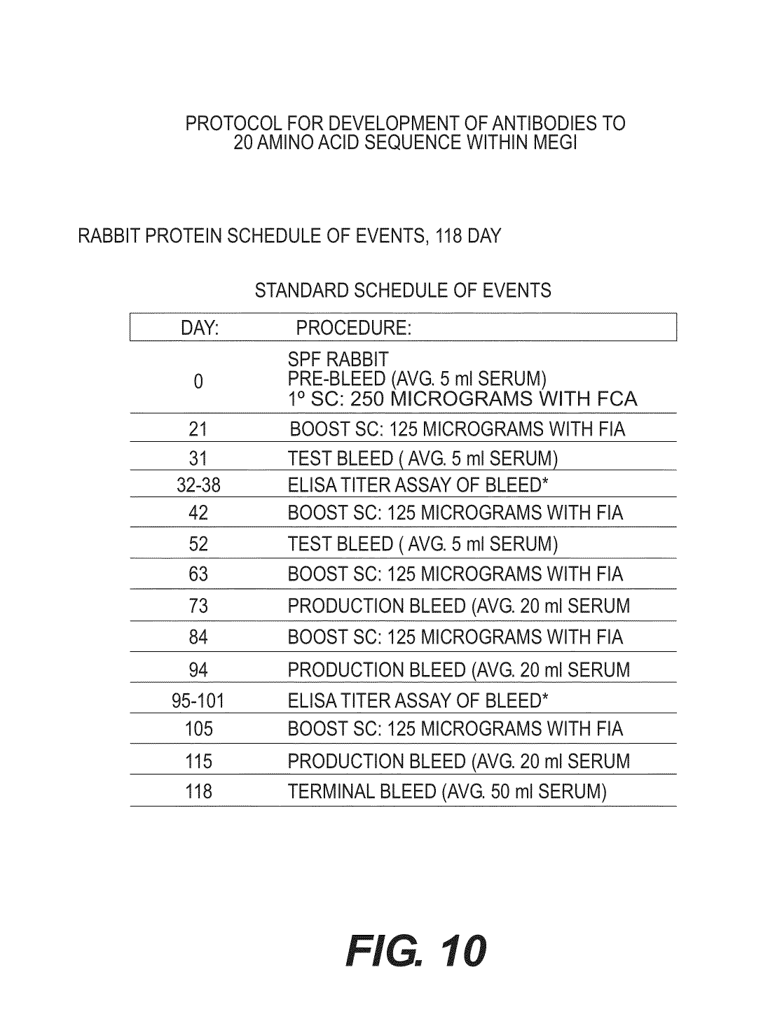

The middle images (FIG. 8b and FIG. 8e) are cells grown in SFM. Megi is localized in the cytoplasm, as indicated by cytoplasmic Cy3 staining. The arrows in FIG. 8b and FIG. 8e show the lack of staining in the position of the nuclei. The arrows in FIG. 8e of cells grown in SFM demonstrate intense blue DAPI staining (shown by light grey contrast) of the nuclei, indicating a lack of Megi in the nucleus. In the upper image (FIG. 8c) showing cells grown in SFM and Reg peptide, the presence of Megi by immunostaining is seen in the nucleus and indicated by the arrows in FIG. 8c and demonstrates a translocation of Megi into the nucleus. In the lower image (FIG. 80, there is an overlap of Megi-Cy3 staining and nuclear DAPI staining that corroborates that there is nuclear localization of Megi indicated by the arrows in FIG. 8f. In the lower image (FIG. 8f) of cells grown in SFM and Reg peptide, the arrows indicate the position of the nuclei, which now stains for Megi. Scale bar=20 μm in all images.

FIG. 9 shows the results of Enzyme ImmunoAssay studies measuring titers from peptide sequences within SEQ ID NO: 11.

FIG. 10 demonstrates the standard protocol used for development of polyclonal antibodies to peptide regions within Megi.

### DETAILED DESCRIPTION OF THE INVENTION

The present invention identifies that Megi is: (1) a protein expressed on human pancreatic tumors; (2) critical in the signaling pathway of pancreatic cancer growth and expansion; (3) identified as a new serum and tissue biomarker that is present in several types of tumors that can be evaluated in tissue utilizing a monoclonal anti-human MEGI antibody and can be identified in plasma utilizing quantitative reverse transcription-polymerase chain reaction; and (4) a key receptor in the signaling, pathway of tumor growth and expansion in a number of human cancers including pancreatic adenocarcinoma.

Further, the present invention shows that the inhibition of Megi and/or competition to binding sites on the Megi receptor limits signaling and/or blocks downstream signaling pathways of the Megi protein, resulting in diminished and limited tumor expansion and growth in cancers expressing Megi including, but not limited to, pancreatic cancer, colorectal adenocarcinoma, primary adenocarcinoma of the liver, squamous cell carcinoma of the esophagus, gastric carcinoma, head and neck and tumors of the prostate, bladder and breast cancers and other tumors in which Megi may be detected on a cancer cell surface.

Additionally, the present invention shows that inhibitors of Megi and its downstream pathway serve as adjunctive therapy for pancreatic, colorectal, bladder, primary hepatocellular, prostate, gastric carcinoma, breast carcinoma and other tumors. The present invention further identifies inhibitors of Reg1α, Reg3α and Megi block downstream tumor trophic factors in cancer. The present invention also identifies a highly conserved region of Reg1α, Reg3α and other Reg proteins that interacts with the Megi receptor binding site and is, therefore, and is a target for inhibitory molecules of this homologous region on the identified Reg proteins that interact with Megi that may also be developed as a therapeutic vaccine and preventive vaccines in populations at risk. The invention also identifies the binding site for Megi, and identifies sequences within Megi for successful development of inhibitory antibodies and potential therapeutic and preventive vaccine, which are target sites for inhibiting the downstream reaction triggered by the Megi-Reg interaction.

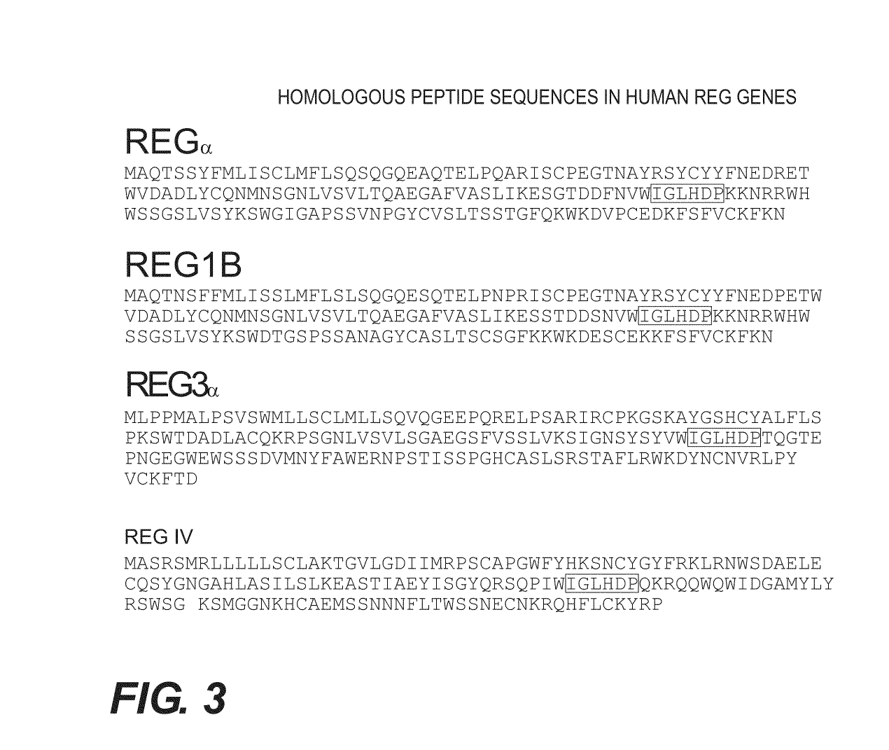

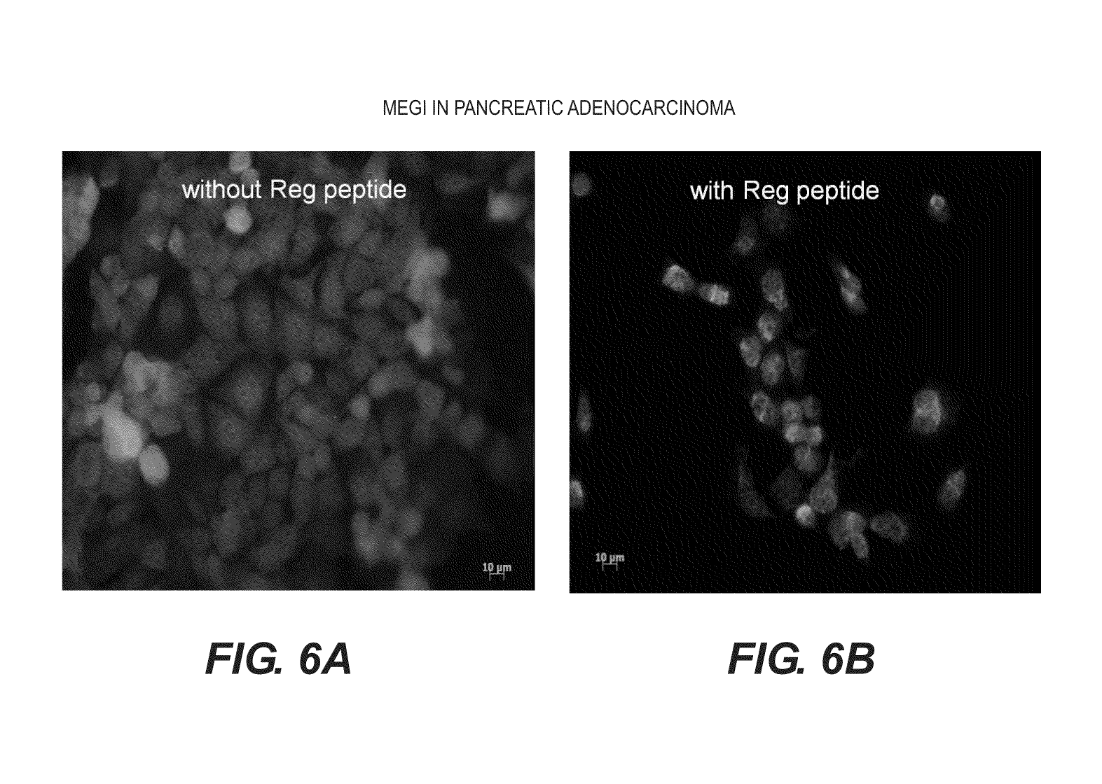

To date, there has been no data demonstrating showing that Megi is present on adenocarcinoma cells and is a receptor for Reg ligands as a receptor on tumors. Data presented demonstrates that the tumor activity in increased by Reg peptide acting through Megi resulting in more rapid translocation of Megi from the cell surface to the nucleus of tumor cells. Neither Megi, Reg1α nor Reg3α have been shown as primary factors in the process of tumorigenesis. As discussed in the Examples below, the present invention identifies the three-dimensional structure of Reg1α and Reg3α and identifies a common structure in each (and in other members of the Reg family) that is the site for the reaction between Reg and Megi. FIGS. 3 and 6 show the three dimensional representations of Reg and Megi, with the transmembrane receptor highlighted. Without being restricted to any particular theory, it is believed that the binding of Reg to Megi results in translocation of Megi from the cell membrane into the nucleus of the cell, stimulating differentiation of new cells and cell growth and resulting in increased levels of TNFα and NF-kβ in human adenocarcinoma.

There have been no, prior studies illustrating that the pathophysiological role of Reg1α, Reg3α and Megi are directly mediating tumor growth and expansion. To date, it has been hypothesized that Reg1α and Reg3α are upregulated secondary to inflammation, but do not catalyze downstream tumor activity. Reg1α and Reg3α have only been hypothesized to be markers rather than playing a direct role in cancer cell progression.

NF-kβ and TNFα are a rapidly-acting transcription factor to harmful stimuli. The known inducers of NF-kβ activity are highly variable. Dysregulation of NF-kβ has been linked to cancer and inflammatory with similar findings for TNFα and other proinflammatory cytokines. Q. Li et al., (2002) Nature Reviews Immunology 2: 725-734, H. Qin et al, (2005) Blood 106 (9): 3114-3122. This data in this invention are supported by the work of Mizuno and colleagues that overexpression of Megi enhances NF-kβ activity induced by TNF-alpha. K. Mizuno et al., Cell Signal (2001)13(2):125-3.

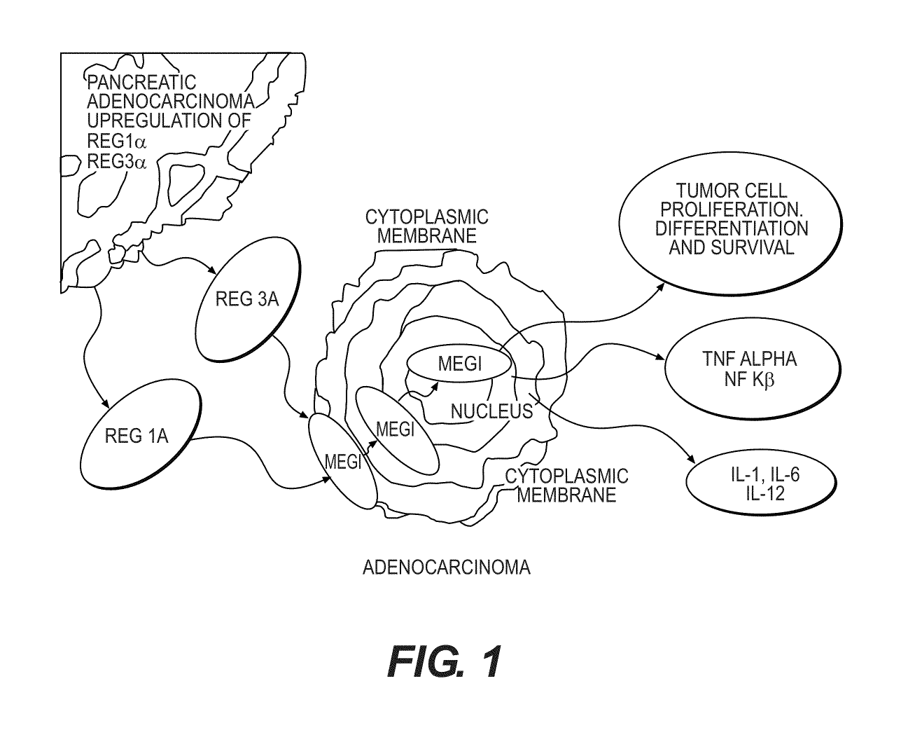

The findings herein also define Megi as a Reg receptor in human cancer with data suggesting that Reg1α and Reg3α overexpression in tumor states results in activation of Megi, which may result in increased and unregulated NF-kβ and TNFα. These findings are shown schematically in FIG. 1.

Therefore, based on the findings of the present invention, inhibitors, antisense constructs and other molecules are being designed to target Megi, Reg1α and Reg3α (and other Reg molecules such as Reg1β and RegIV) to treat cancer and reduce the size of tumors and also include a therapeutic vaccine, which may potentially be used as a preventive vaccine in populations at risk. In another embodiment of the invention, inhibitors, antisense constructs and other molecules can be designed to target the amino acid region common to many Reg proteins, IGLHDP (SEQ ID NO: 9), as shown in FIG. 3 and sequences within peptides 1-332 in Megi (SEQ ID NO: 1) and specifically to a 20 amino acid sequence (SEQ ID NO: 4) for which an antibody has been developed (as described more specifically in the Examples).

The present invention also includes markers for novel screening and monitoring methods for cancer, for example, pancreatic cancer. To date, there are no serum tumor markers for pancreatic cancer. Tumor markers such as Ca-19-9 and CEA have not been helpful in diagnosis, treatment or improving the longevity and quality of life for patients with adenocarcinoma of the pancreas. Based upon the lack of true tumor markers for pancreatic adenocarcinoma, new screening and monitoring methods based upon this invention utilizing plasma and tissue levels of Reg1α, Reg3α and Megi are presented reflecting the significant and pathological role of Reg 1a and Reg3α and their interaction with the Megi receptor on human cancer cells.

In a further embodiment of the invention, Reg1α, Reg3α and Megi are plasma markers utilized as screening tools for early detection of pancreatic and other cancers in high risk populations, such as patients with diabetes, chronic pancreatitis and family history of pancreatic and other tumors. Pancreatitis and diabetes are two clear risk factors for pancreatic cancer. Both type 1 and type 2 diabetes have been linked to pancreatic adenocarcinoma. The diagnosis of diabetes is present long before the diagnosis of pancreatic cancer is made. As many of 80% of all patients diagnosed with pancreatic cancer have a disorder of glucose metabolism. Wang F., Molecular Cancer (2003) 2:4; Pannala R Lancet, Oncol. (2009); 10(1):88-95.

The relationship of diabetes, which is an endocrine disorder, and pancreatic cancer, which is almost always an exocrine ductal carcinoma, had not previously been identified. Recently, there has been new literature on the relationship between exocrine pancreatic tissue and endocrine islets with findings to substantiate that new endocrine islets form postnatally from exocrine ducts. Levetan C, Journal of Diabetes (2010), 2(2):76-84, Bonner-Weir S, Pediatric Diabetes (2004), 5:16-22. It has been shown by several teams in both animal and humans that Reg3α is upregulated in type 1 diabetes. See, e.g., Vukkadapu S S., Physiol Genomics (2005), 21:201-211; Gurr W, Diabetes. (2002), 51(2):339-46; Zenilman M E, Pancreas (1998), 17(3):256-6.

Further, based upon the literature and findings that Pancreatitis Associated Protein is Reg3α, there is now a clear relationship between the upregulation of Reg3α and Reg1α as the body's mechanism to protect against diabetes. With chronic upregulation of Reg1α and Reg3α in diabetes, chronic and acute pancreatitis, and during pancreatic stones, the present invention discloses that there is an increased'potential for dysregulated and unregulated cell populations of Reg1α and Reg3α to become autonomously functioning resulting in a neoplasm in patients with diabetes, pancreatitis and with pancreatic stones.

Because the literature finds that about 80% of patients diagnosed with pancreatic adenocarcinoma have glucose intolerance or frank diabetes (F. Wang, Molecular Cancer (2003), 2:4) development of plasma markers identifying Reg1α, Reg3α and Megi may lead to earlier diagnosis, treatment, earlier surgery and potential cure for pancreatic adenocarcinoma and potentially other tumors expressing Reg1α and Reg3α. Tumor markers both in tissue and plasma utilizing the overexpression of Reg1α, Reg3 α, and Megi in at risk populations at risk, such as those with diabetes, have the potential to diagnose and treat cancers, such as adenocarcinoma of the pancreas, earlier and with better outcomes than now exist.

To date, there is no successful serum screening for a variety of tumors including pancreatic cancer. Plasma screening known in the art includes Carcinoembryonic antigen (CEA), Alpha-Fetoprotein (AFP), CA19-9, CA-125, but there have been no adequate serum tumor markers for earlier identification of pancreatic cancer, hepatocellular cancer, and other adenocarcinomas, which may specifically have Megi as a rate-limiting receptor for activation and acceleration by Reg proteins. Based on the invention described herein, new plasma screening methods measuring Megi, Reg1α, Reg3α are developed utilizing RT-PCR for earlier diagnosis and treatment monitoring of tumors expressing MEGI. The findings in this invention are in contradiction to Motoo (Int J Pancreatol. 1998; 23(1):11-6), who has previously demonstrated the ability to measure Reg in serum, and specifically stated that there was no relationship of Reg to tumor pathology (Int J Pancreatol. 1998; 23(1):11-6). It is hypothesized based on the data of the present invention that Megi is a key tumor receptor site and that upregulation of Reg gene proteins are pathological and interact with Megi on the cytoplasmic membrane of adenocarcinoma of the pancreas stimulating more rapid movement of Megi to the cell nucleus leading to tumor spread and activity.

Supporting this hypothesis is recent data in which Reg has been shown to act directly through the Megi receptor in the rat Reg Hippocampus (Van Ba J., et al., Biol Chem. 2011 Dec. 9 (published electronically at www.jbc.org/cgi/doi/10.1074/jbc.M111.260349). Van Ba found that when Reg1α is upregulated, there is over-expression of Megi, and with the use of recombinant Reg1α on rat Reg hippocampus, there is neurite outgrowth. Reg1α was found to be ineffective in neural growth when Megi over-expression was down-regulated by shRNA. These findings indicate that the Reg1α effect is mediated by through the Megi receptor, and when Megi is blocked, the Reg1α impact on neuronal growth is limited. (Van Ba J., et al., Biol Chem. 2011 Dec. 9, available at www.jbc.org/cgi/doi/10.1074/jbc.M111.260349 Epub ahead of print).

Our data also illustrates that the Megi receptor in cancer cells, is a pivotal receptor and therapies aimed at blocking Megi will limit stimulation by Reg and limit downstream cellular speed of growth and activity.

The ability to utilize mRNA from Reg1α or Reg3α and Megi as serum markers in tumors for earlier diagnostic purposes and for utilization of reverse transcription polymerase chain reaction for the exponential amplification via reverse transcription polymerase chain reaction provides a highly sensitive technique in which a very low copy number of RNA molecules can be detected. In contradistinction to the report by Motoo, who was able to detect in plasma levels of Reg3α, also known as PAP among 20% of patients with gastrointestinal tumors, but specifically stated that they did not find or report any relationship between Reg3α and cancer (Int J Pancreatol (1998); 23(1):11-6), the findings in this invention demonstrate that detectable plasma levels of Reg1α or Reg3α are significant and part of the pathological process in cancer.

In another embodiment of the present invention, inhibition of Megi signaling results in tumor inhibition and deceleration of signaling of Megi from the cell membrane to its downstream signaling cascade, resulting in diminished tumor expansion leading to improved morbidity, mortality and quality of life among patients suffering with tumors that express Megi, including pancreatic, colorectal, adenocarcinoma, primary adenocarcinoma of the liver, squamous cell carcinoma of the esophagus, head and neck and tumors of the prostate, bladder, and also reduce expansion of breast and other tumors in which MEGI is a candidate gene.

In another embodiment of the present invention, serum biomarkers may be developed for early detection of pancreatic and other tumors. Tumor markers for pancreatic cancer such as Ca-19-9 and CEA have not been helpful in diagnosis, treatment or improving the longevity and quality of life for patients with adenocarcinoma. The ability to screen via plasma would provide a great breakthrough for such populations at risk. Based on findings that Reg1α or Reg3α may accelerate tumor growth and spread, and both Reg1α and Reg3α are not expressed by normal functioning tissue, usage of plasma identification of Reg1α or Reg3α as developed may be extremely useful for earlier identification of patients with neoplasm. The presence and overexpression of Megi in tumors provides a very unique serum marker for occult neoplasms and may specifically be utilized as a tumor marker and therapeutic marker when inhibitory agents being developed for Megi are utilized.

In order to antagonize the binding of either the ligand or receptor, an antibody can be used. The term “antibody” as used herein includes monoclonal and polyclonal antibodies, as well as antibody portions or derivatives that contain the variable region of an antibody. Such portions or derivatives include, but are not limited to, single chain Fv (ScFv), Fab, F(ab′)₂, and Fv. Also included are conjugates that present the variable region on a framework of another molecule. The antibody specifically binds to a Reg1α or Reg3α ligand, or to the Megi receptor, preferably binding far less to other unrelated proteins. The binding to a desired Reg family target or Megi and to other proteins preferably differs by at least a factor of 10², 10³, 10⁴, 10⁵, or 10⁶. Antibodies have long been administered to humans for a variety of purposes. Any method of administration known in the art can be used in the context of the present invention, including but not limited to intravenous, intraperitoneal, subcutaneous, intramuscular, intranasal, intrabronchial, intratumoral, and per oral administration.

Further embodiments of the present invention provide methods for administering the antibodies, antisense, RNA interference, small molecule inhibitors alone or in combination with other therapeutic agents including a therapeutic antigen/adjuvant vaccine derived from Megi, REG1α or REG3α (or other members of the REG family) to be utilized with other techniques for chemotherapeutic medications, surgery, radiation and other strategies for inhibiting pancreatic cancer and other tumors expressing REG 1 α, REG 3 α, or MEGI.

In various embodiments of the present invention, the methods of the invention can be practiced by administration of a therapeutically effective inhibition amounts that competitively bind to, limit expression of, inhibit or limit the activity of the Megi receptor, Reg1α or Reg3α, or other members of the Reg family, or to the homologous amino acid sequences in Reg1α, Reg1β, Reg3α and Reg IV alone, or in combination with other chemotherapeutic agents, radiation and biological agent, vaccines, antiangiogenic agents and anti-tumor and tumoricidal agents including but not limited to 5-fluorouracil (5-FU), sorafenib, gemcitabine, erlotinib, capecitabine, tegafur oxaliplatin, irinotecan, leucovorin, levamisole, bevacizumab, epidermal growth factor receptor-targeted monoclonal antibodies, cetuximab panitumumab, fluoropyrimidines, nucleoside cytidine analogues, platinum analogues, topoisomerase inhibitors, antimicrotubule agents, proteasome inhibitors, vitamin D analogues, arachidonic acid pathway inhibitors, histone deacytylator inhibitors, farnesyltransferase inhibitors, epidermal growth factor receptor therapies and other chemotherapies, including other immunomodulating agents and monoclonal antibodies directed at TNFα, Interleukins 1 beta and alpha, IL-6, IL-9, IL-12, IL-20 and other agents utilized in cancer treatment.

Additionally non-steroidal anti-inflammatory drugs (NSAIDs) and COX-2 inhibitors in particular can be administered to the tumor cell in connection with the agents of the present invention. Such drugs include indomethacin, naproxen, ibuprofen, naproxen, aspirin, celecoxib, diclofenac, etodolac, fenoprofen, ketoprofen, ketoprofen, ketoralac, oxaprozin, nabumetone, sulindac, tolmetin, valdicoxib, Bextra®, rofecoxib and other anti-inflammatory and immunomodulating agents. These can be administered simultaneously with the antibodies or before or after, preferably within 1 day, 1 week, or 1 month of the antibodies.

Therapeutic radiation can also be administered to the same tumor cell (or if in a patient, to the same cancer patient). Similarly anti-cancer chemotherapeutic agents can be administered to the same tumor cell or cancer patient. Such agents include: x-rays, cisplatin (Platinol®), daunorubicin (Cerubidine®), doxorubicin (Adriamycin®), etoposide (VePesid®)), methotrexate (Abitrexate®), mercaptopurine (Purinethol®), fluorouracil (Adrucil®), hydroxyurea (Hydrea®), Vinblastine (Velban®), Vincristine (Oncovin®), Irinotecan (Camptosar®, CPT-11), Levamisole, selective epidermal growth factor receptor tyrosine kinase inhibitors (e.g. ZD1839, Iressa®) and Pacitaxel (Taxol®). Preferably the agents co-administered with the antibodies are ones that induce apoptosis.

The types of tumors and tumor cells that are good targets for the treatments of the present invention include all of those tumors in Megi, Reg1α or Reg3α or other Reg proteins are present on tumor tissue or elevated in plasma testing and may include the non-squamous including adenocarcinomas of the pancreas, gastrointestinal tract (e.g. esophageal, gastric, small intestine, colon and rectal adenocarcinomas), squamous cell tumors of the esophagus, the hepatobiliary tract including cholangiocarcinoma, primary liver carcinoma and the pancreas. Suitable tumor targets may also include ovarian, brain, prostate, breast, germ cell tumors, papillary serous carcinoma, lung carcinoma, non-small cell tumors of the lung, acute myelogenous leukemia, B-cell chronic lymphocytic leukemia, insulinomas, fibrosarcoma, juvenile granulosa tumor cells and tumors in which there is expression of Reg1α, Reg3α (or other Reg proteins) or Megi in tissue or in plasma. Other tumor cells, especially those which express REG1α or REG3α (or other members of the REG family) and MEGI can be targeted.

Similar to the antibody-type therapeutic agents, antisense constructs, small interfering RNAs (siRNAs), antisense oligonucleotides, and RNA interference constructs can be used to increase apoptosis in cancer cells. These agents function by preventing or interfering with expression of REG1a and REG3a (and other REG genes) and MEGI in order to inhibit downstream signaling. The use of such agents is well known in the art and also includes therapeutic vaccines acting as antigens resulting in immune inhibition of cancer.

Antisense constructs, antisense oligonucleotides, RNA interference constructs or siRNA duplex RNA molecules can be used to interfere with expression of any of the members of the REG family, for example REG 1α and REG 3α, and MEGI. Typically at least 10, 15, 17, 19, or 21 nucleotides of the complement of the selected mRNA sequence are sufficient for an antisense molecule. Typically at least 10, 19, 21, 22, or 23 nucleotides of the selected RNA are sufficient for an RNA interference molecule. Preferably an RNA interference molecule will have a 2 nucleotide 3′ overhang. If the RNA interference molecule is expressed in a cell from a construct, for example from a hairpin molecule or from an inverted repeat of the desired REG gene family sequence, then the endogenous cellular machinery will create the overhangs. siRNA molecules can be prepared by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, RNA 7: 1509-1521: Hutvagner G et al., Curr. Opin. Genetics & Development 12: 225=232; Brummelkamp, 2002, Science 296: 550-553; Lee N S, Dohjima T, Bauer G, Li H, Li M-J, Ehsani A, Salvaterra P, and Rossi J. (2002). Nature Biotechnol. 20:500-505; Miyagishi M, and Taira K. (2002). Nature Biotechnol. 20:497-500; Paddison P J, Caudy A A, Bernstein E, Hannon G J, and Conklin D S. (2002). Genes & Dev. 16:948-958; Paul C P, Good P D, Winer I, and Engelke D R. (2002). Nature Biotechnol. 20:505-508; Sui G, Soohoo C, Affar E-B, Gay F, Shi Y, Forrester W C, and Shi Y. (2002). Proc. Natl. Acad. Sci. USA 99(6):5515-5520; Yu J-Y, DeRuiter S L, and Turner D L. (2002). Proc. Natl. Acad. Sci. USA 99(9):6047-6052.

Antisense or RNA interference can be delivered in vitro to turn or cells or in vivo to tumors in a mammal. Typical delivery means known in the art can be used. For example, delivery to a tumor can be accomplished by intratumoral injections. Other modes of delivery can be used without limitation, including: intravenous, intramuscular, intraperitoneal, intra-arterial, subcutaneous, and per oral, delivery. Conversely in a mouse model, the antisense or RNA interference can be administered to a tumor cell in vitro, and the tumor cell can be subsequently administered to a mouse. Vectors can be selected for the desirable properties for any particular application. Vectors can be viral or plasmid. Non-viral carriers such as liposomes or nanospheres can also be used.

Additional therapeutic agents or candidate therapeutic agents can be identified using a binding assay. The assay can be performed in vitro using purified proteins, or using whole cells. A Reg protein, for example Reg1α or Reg3α, is contacted with Megi in the presence and absence of a test substance. The order of the contacting can be varied. All components can be, but need not be simultaneously contacted. Pairs of reagents can be pre-bound, for example, and displacement by a third reagent can be assessed. The test substances can be natural products or synthetic products; they can be single compounds or combinations of compounds. They can be known or unknown for another use. They can be pre-selected based on structural similarities to other compounds, or they can be screened randomly. Any binding assay known in the art can be used. A two-hybrid type assay can be used in which expression of a reporter gene is dependent on the binding of two binding partner proteins. Alternatively, one of the binding partners may be bound to a solid support. One binding partner can be labeled. Bound or unbound, complexes can be separated from the assay mixture; for example, using immunoprecipitation. Another assay utilizes cells which express one of the binding partners on their cell surface and the other binding partner is added exogenously. Any format for assessing binding between two proteins can be used. Once a binding inhibitor of the Reg protein, for example Reg1α or Reg3α, and the Megi receptor has been identified, it can be used therapeutically to treat tumor cells and tumors. The inhibitor can be administered by any route, particularly intravenous, intramuscularly, subcutaneously, and per mouth.

Other embodiments of the present invention provide pharmaceutical formulations and unit dose forms of inhibitors and competitors of Megi and its downstream pathway. In one embodiment, the pharmaceutical formulation provided contains the antisense antibody of Megi alone or in combination with one or more other active pharmaceutical ingredients (APIs).

In one embodiment of the present invention, the API is an agent in soluble liposome preparations that allow the optimized delivery to be administered by a variety of routes, including subcutaneously, intramuscularly, intravenously, and orally, depending on the formulation selected.

The formulation is for general systemic administration, but in other embodiments, the formulation of therapy for inhibition of Reg proteins, for example Reg1α or Reg3α, or Megi is specifically developed for targeted administration of therapy to pancreas specific organ, organ system, location, receptor, cells, tissues, organs, or organ systems within a subject, which includes specific targeting of the pancreas, liver or other organs via encapsulation of therapy including, but not limited to a lipid bilayer with a sialic acid residue and/or other specific targeting incorporated into lipid bilayer for more efficacious delivery of a Reg or Megi inhibitor to a specific location of the cancer or tumor.

In other embodiments of the present invention, provided are methods of treating a pathology associated with pancreatic cancer and other cancers expressing REG (for example REG 1α or REG 3α), or MEGI, and among those tumors in which such genes are candidates, in a subject in need of such treatment. The method may comprise the step of administering one or more agents or radiation for inhibiting tumor cell growth. In one aspect of this embodiment, the agents are selected from including but not limited to 5-fluorouracil (5-FU), sorafenib, gemcitabine, erlotinib, capecitabine, tegafur oxaliplatin; irinotecan, leucovorin, levamisole, bevacizumab, epidermal growth factor receptor-targeted monoclonal antibodies, cetuximab panitumumab, fluoropyrimidines, nucleoside cytidine analogues, platinum analogues, topoisomerase inhibitors, antimicrotubule agents, proteasome inhibitors, vitamin D and vitamin D analogues, arachidonic acid pathway inhibitors, histone deacytylator inhibitors, farnesyltransferase inhibitors, epidermal growth factor receptor therapies and other chemotherapies. Other agents to be added include COX-2 inhibitors.

In another embodiment of the present invention, methods of treating a pathology associated with impaired pancreatic function in a subject in need of such treatment are provided. The method may comprises one or more of the steps of limiting tumor cell growth and expansion by administering injections or oral usage of ascorbic acid in dosages as high as 4 grams/kg per day body weight, Vitamin B6, B12, folate and Vitamin D2 (ergocalciferol), D3 (cholecalciferol) and/or 1, 25 Dihydroxyvitamin to maintain 25-hydroxyvitamin levels above 40 ng/ml

### EXAMPLES

To evaluate the role of Reg1α, Reg3α and Megi in pancreatic cancer, human adenocarcinoma ductal cells lines were utilized to determine the presence of Megi in active cancer cells.

Studies were designed to evaluate the following:
1) whether Megi is present on human adenocarcinoma of the pancreas;
2) whether Megi is a Reg1α and/or Reg3α receptor on pancreatic adenocarcinoma cells;
3) whether Reg1α and/or Reg3α interact with Megi and have an impact on downstream signaling;
4) whether Reg1α and/or Reg3α enhance signaling speed and downstream trafficking in adenocarcinoma; and
5) whether Reg1α and Reg3α modulate expression and subcellular localization of Megi in adenocarcinoma of the pancreas

### Example 1

### Determining the Presence of Megi on Adenocarcinoma of the Pancreas

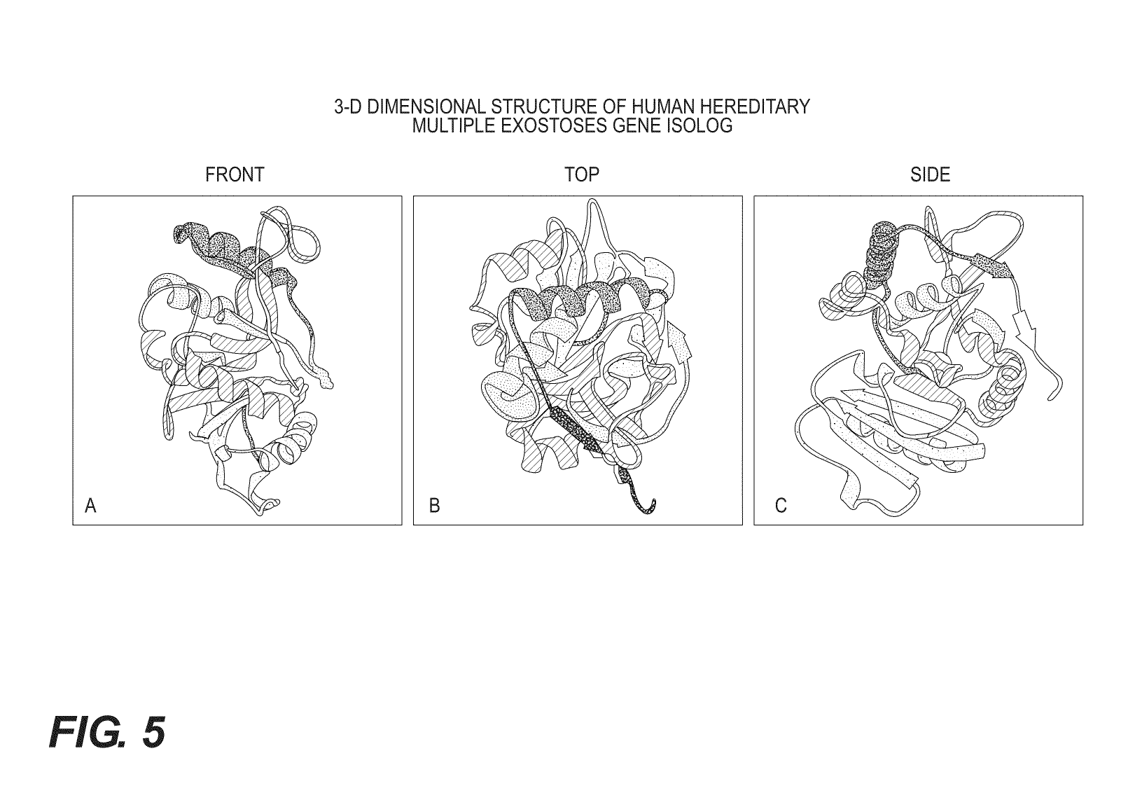

The primary candidate protein to potentially to rate-limiting in adenocarcinoma of the pancreas was identified as Megi (FIG. 5). This investigation was undertaken to determine if Megi was present on adenocarcinoma of pancreas and whether Reg peptide modulates expression of Megi and subcellular localization. There has been no previous description of Human Hereditary Multiple Exostoses Isolog gene protein (Megi) specifically playing a role in adenocarcinoma. Megi was selected as a possible receptor on pancreatic tissue because it was found as a binding protein for Reg1 in rats (Kobayashi S., et al., J Biol Chem 2000, 275, 10723-10726).

This investigation was undertaken to determine if Human Hereditary Multiple Exostoses Isolog gene protein (Megi) was present on adenocarcinoma of pancreas and whether Reg peptide modulated expression and subcellular localization. The initial study evaluated the presence of Megi in human pancreatic adenocarcinoma cells followed on the impact that Reg peptide may have in tumor cells and its relationship to Megi.

To determine the presence of Megi via immunofluorescence, a continuous tumor-cell line from a human carcinoma of the exocrine pancreas were cultured in Dulbecco's Modified Eagle Medium containing 4.5 g/L glucose, 1:5 g/L sodium bicarbonate, 4 mM L-glutamine, and 10% fetal bovine serum. Cells were seeded at 50% confluence in standard growth medium. Twenty-four hours after seeding, cells were differentiated in serum free medium. Cells were mounted on cover slips and fixed in 4% paraformaldehyde and permeabilized with 0:2% Triton X-100. After being washed and blocked, cells were incubated with rabbit polyclonal anti-Megi antibody and a CY3-conjugated goat-anti-rabbit labeled fluorescent antibody. Antibodies were used at a 1:200 dilution. Coverslips were mounted and staining was analyzed on a Zeiss Axiovert 220M microscope powered by Axiovision 4.0 software with Z-stack acquisition, 3D-deconvolution, and 4D rendering modules.

Utilizing Cy3 immunofluorescent staining of Megi in human pancreatic adenocarcinoma cells in standard medium, there is immunofluorescent staining of Megi (FIG. 6a), which is well-defined at the cell borders indicating surface expression of Megi on the plasma membrane with visibly undetectable levels of Megi in the nucleus. Cells were found (FIG. 6a) that the cell borders were illumined with Megi with well-defined surface expression of Megi on the cell surface with visibly undetectable levels of Megi inside of the cells. The observation was made that Megi protein is expressed in human pancreatic cancer cells and clearly visible on the cytoplasmic membrane of pancreatic adenocarcinoma cells.

### Example 2

### Megi as Reg Protein Receptor on Tumor Cells

Previous studies have found that Reg proteins are expressed and is a tumor marker in human cancer, but to date, Reg1α or Reg3α have been considered inflammatory makers and there has been no direct relationship made between Reg1α or Reg3α playing a role in tumor growth. Many teams have shown that Reg mRNA is undetectable in sera, pancreatic secretions and pancreatic tissue in normal healthy adults. No direct role of Reg1α or Reg3α has been shown to play in stimulating tumor cells or enhancing tumor cell growth, speed or downstream tumor pathways. These studies were undertaken to determine if the presence of Reg1α or Reg3α may impact its potential receptor (Megi) in pancreatic cancer cells and have an impact on cancer.

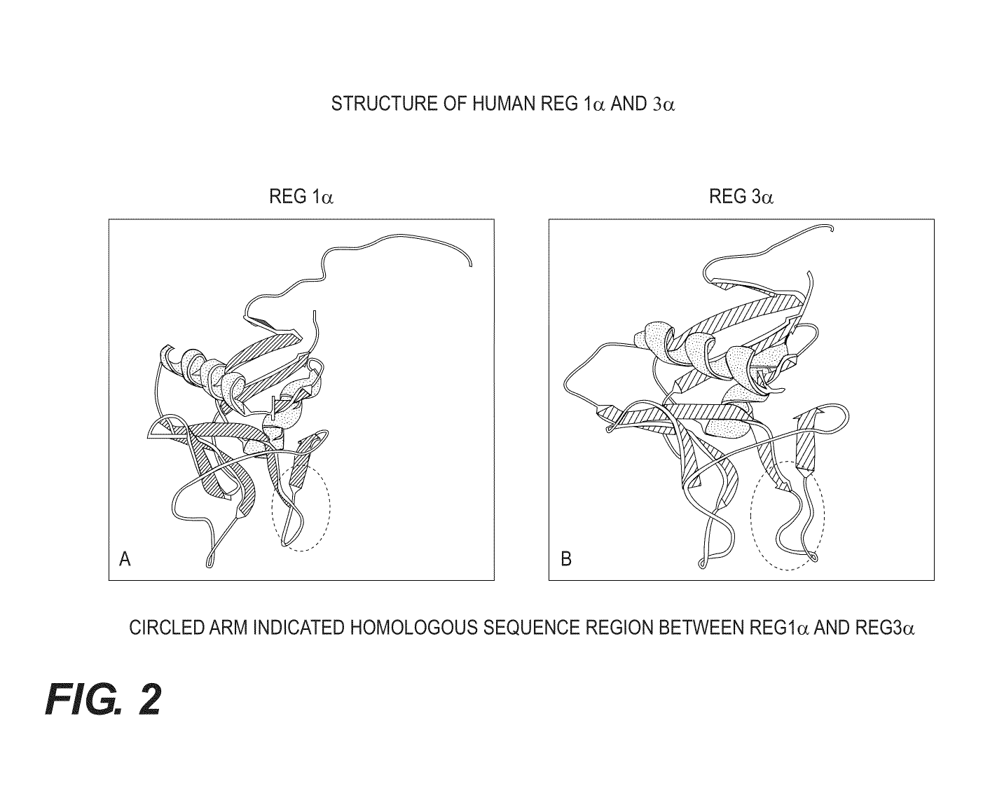

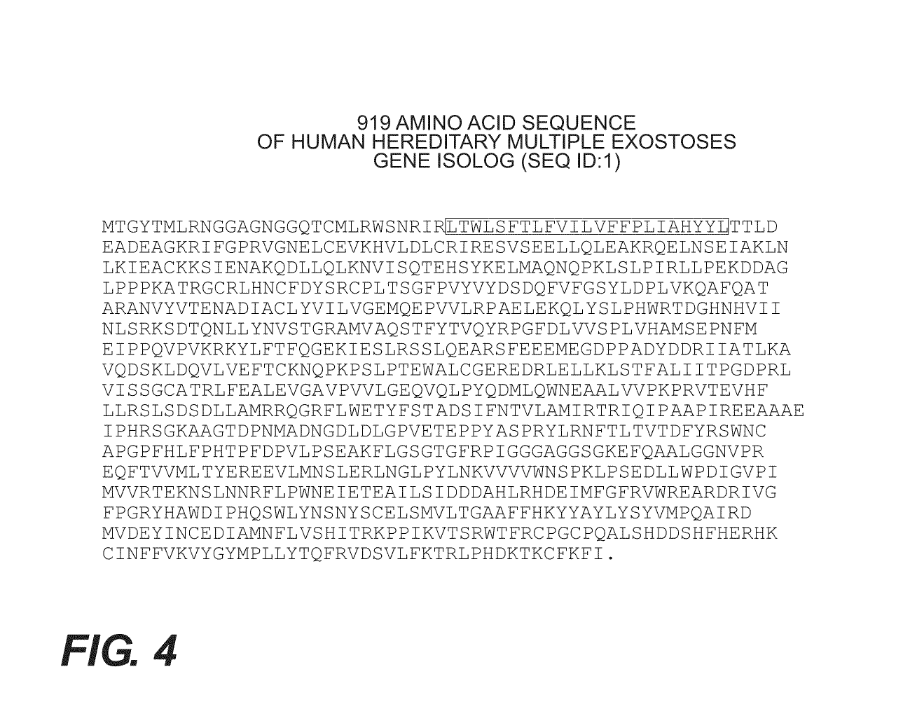

By evaluating the three-dimensional structures of Reg1α and Reg3α derived from the primary sequences and folded by SwissProt folding algorithms with the predicted three-dimensional structure of both peptides was similar and is shown in FIG. 2. FIG. 2a shows the three dimensional structure for Reg1α and FIG. 2b shows the three dimensional structure for Reg 3α. When both Reg1α and Reg3α are aligned by amino acid sequence (FIG. 4) and the three-dimensional structures are overlaid with the amino acid sequences, both Reg1α and Reg3α are found to have a common signaling region, which is present in a right protrusion (circled in FIG. 2), in which there is located an identical peptide sequence,

The alignment (FIG. 3) of the 166 amino acid sequence for Reg1α (SEQ ID NO: 5), the 166 amino acids of Reg1β (SEQ ID NO. 6), the 175 amino acid sequence for Reg3α (SEQ ID NO: 7) and the 158 amino acid sequence for RegIV (SEQ ID NO: 8) was studied. Based upon the structure and 3-dimensional folding and properties of the amino acids of these Reg proteins, a common peptide sequence (SEQ ID NO: 10) was selected and synthesized by Bachem (www.bachem.com) to evaluate the potential impact of a Reg peptide on human adenocarcinoma. This area, of the sequence is hypothesized to be a key signaling domain for Reg1α and Reg3α (and other Reg proteins) to a common Megi receptor.

When (167 nM) Reg peptide was added to human pancreatic adenocarcinoma cells, there was a significant visible difference (FIG. 6b) in cellular staining of Megi. There is greater expression of Megi inside of the cell with less cytoplasmic membrane expression seen on the surface of cells when Reg peptide is added to the medium. To further confirm that Reg peptide had an impact on the Megi receptor, further studies were conducted to confirm and evaluate the impact of Reg on Megi and any enhancement in cellular trafficking that was directly attributable to the interaction between Reg and Megi.

### Example 3

### Enhancement in Tumor Activity and Speed

Western blot analyses were utilized to evaluate and confirm the impact of Reg on Megi translocation from the cytoplasmic membrane, through the cytoplasm and to the nucleus and to determine if there was a measurable enhancement in tumor trafficking when Reg peptide is added to cancer cells.

The Western blot analysis of cytosolic and nuclear fractions isolated at the indicated time points with Reg peptide accelerating time of Megi translocation from the cell surface membrane to nucleus. Adenocarcinoma cells were seeded in T75 flasks in Dulbecco's Modified Eagle media containing 10% fetal bovine serum. The cells were incubated at 370 C, 5% CO₂ for 24 hours and then treated with Reg peptide at the final concentration of 167 nM. This treatment was performed once a day for four days. On the fifth day the cells were broken to obtain the cell lysates. In these cell extracts the total protein levels were determined, and 50 micrograms of total protein, were used to perform the western blot analysis. The samples containing 50 micrograms of proteins were diluted in loading buffer and loaded into each well of the gel. For gels run under reducing conditions, buffer also contained 5% of the reducing agent beta-mercaptoethanol

Analyses were performed from cytoplasmic extracts were obtained in 10 mM HEPES (pH 8.0), 1 mM EDTA, 1.5 mM MgCl₂, 10 mM KCl, 0.5 mM DTT, 200 mM sucrose and 0.5% Nonidet P-40. Nuclear extracts were obtained in 20 mM HEPES (pH 7.9), 0.75 mM MgCl₂, 210 mM NaCl, 50 mM KCl, 1 mM EDTA, 10% glycerol, and 0.5 mM of dithiothreitol. Both extraction buffers'contained 0.5 mM PMSF, 1 μg/ml leupeptin, 1 μg/ml aprotinin, 2.5 mM Na₄P₂O₇, 1 mM β-glycerophosphate, and 1 mM Na₃VO₄. Protein extracts were size fractionated on SDS-polyacrylamide gels and transferred to nitrocellulose. After blocking in 3% milk in Tris-buffered saline (pH 7.4), blots were sequentially incubated with rabbit anti-human Megi antibody overnight at 4° C. and appropriate horseradish peroxidase-conjugated secondary antibody. Secondary signals were developed with chemiluminescence substrate and analyzed by autoradiography.

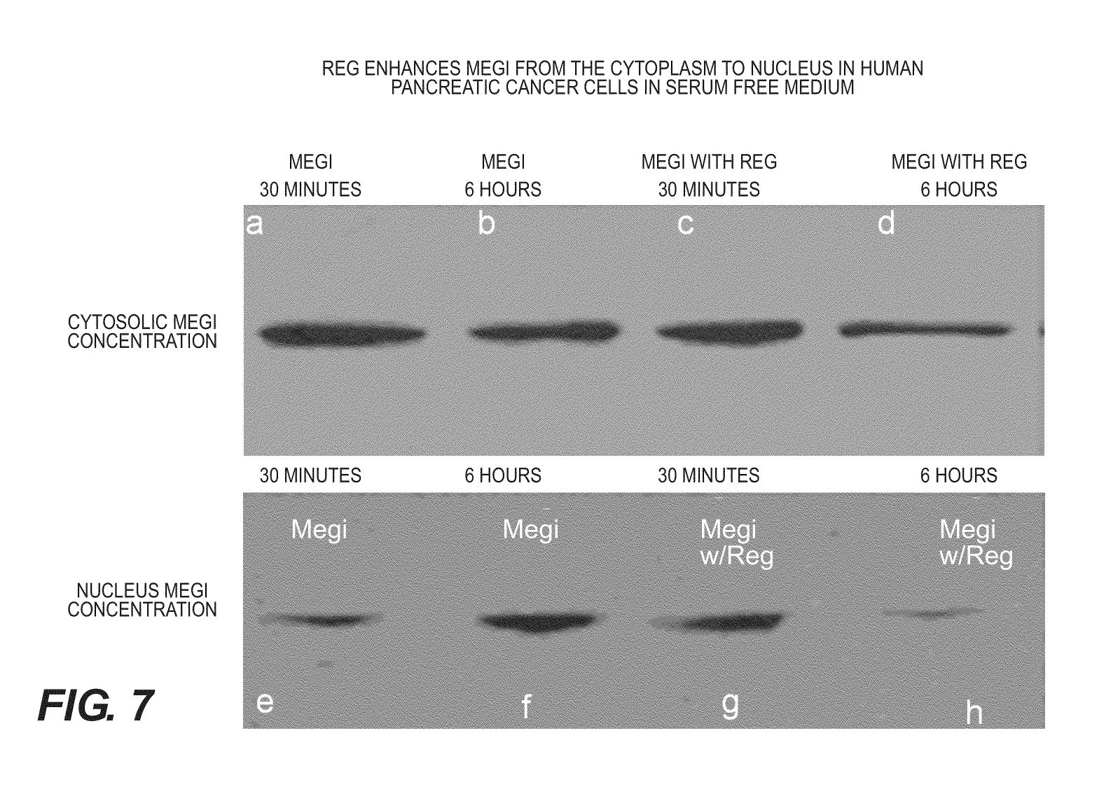

FIG. 7 demonstrates that Western blot analyses of nuclear levels of Megi at time points of 30 minutes and 6 hours with and without the presence of Reg peptide. FIG. 7a demonstrates a significant concentration of Megi in the cytosolic component at 30 minutes with substantially less presence of Megi in the nucleus (FIG. 7e) was observed at 30 minutes. By 6 hours, Megi was observed in the nucleus to a greater extent (FIG. 70 as compared with 30 minutes (FIG. 7e). When. Reg is added to the cancer cells, there is higher nuclear levels of Megi were isolated at 30 minutes (FIG. 7g) compared to nuclear content of Megi in cells not treated with Reg peptide at 30 minutes (FIG. 7e). Addition of Reg into culture media enhanced Megi nuclear translocation and demonstrated by higher nuclear levels of Megi at 30 minutes (FIG. 7g). These comparisons demonstrate that in the presence of Reg, that Megi nuclear translocation time is enhanced and that Reg modulates Megi translocation from the cytoplasmic membrane to the nucleus. Western blot analyses were repeated with results confirming this data.

### Example 4

### Impact of Reg on Megi with Immunofluorescent Nuclear Analyses

FIG. 8 demonstrates the immunofluorescent analyses of Megi demonstrating that Megi moves from cell surface through the cytoplasm and to the nucleus. Immunofluorescent analyses are shown in the upper, panels (FIG. 8a, FIG. 8b and FIG. 8c) indicated by the Cy3 immunofluorescent staining of Megi. In the lower panels (FIG. 8d, FIG. 8e and FIG. 8f) the Cy3 immunostaining of Megi has been overlaid with 4′,6-diamidino-2-phenylindole dihydrochloride (DAPI) staining the nuclei in blue (shown in contrast grey in FIG. 8d, FIG. 8e and FIG. 80. Cells were grown in standard growth medium as a control and compared to cells grown serum-free medium (SFM) in the presence or absence of 150 μM of Reg peptide.

Megi staining is by Cy3 Immunofluorescent red in the upper panels and the lower images include the Cy3 immunostaining of Megi that has been overlaid with DAPI (blue) staining of the nuclei. Cells were grown in standard growth medium as a control (FIGS. 8a and 8d) and compared to cells grown serum-free medium (SFM) in the presence (FIGS. 8c and 8f) or absence of Reg (FIGS. 5b and 8e). The arrows in FIG. 8a and FIG. 8d demonstrate examples of the surface expression of Megi grown in standard growth medium. The cell borders are well-defined indicating surface expression of Megi on the plasma membrane. The arrows delineate the cell borders while the nuclei are shown in contrast grey in FIG. 8d and there is no Megi in the location of nucleus. The middle images (FIGS. 8b and 8e) are cells grown in serum free medium (SFM) and Megi is localized in the cytoplasm as indicated by cytoplasmic Cy3 staining. The arrows in FIG. 5b show the presence of Megi with cell surface expression and expression in the cytoplasm, but with lack of staining in the position of the nuclei as seen by the arrows in the lower image (FIG. 8e) of cells grown in serum free medium without Reg demonstrate intense blue DAPI staining of the nuclei (shown in contrast grey) indicating a lack of Megi in the nucleus. In the upper image of the cells grown in serum free medium and Reg (FIG. 8c), the presence of Megi immunostaining in the nucleus is indicated by the arrows demonstrating a translocation of Megi into the nucleus. In FIG. 8f, the cells are grown in serum free medium and Reg, the arrows indicate the position of the nuclei. In the lower image (FIG. 8f) there is an overlap of Megi-Cy3 staining and nuclear DAPI staining that corroborates the nuclear localization of Megi (shown by the arrows). Scale bar=20 μm in all images.

These findings presented in this invention demonstrate Megi is an active receptor in pancreatic adenocarcinoma cells that can be modulated by Reg peptide. Contrary to previous reports that the presence of Reg1α or Reg3α was not pathological, but rather a reflection of inflammation, we find that Reg stimulates the Megi receptor on cancer cells to move more rapidly from the cytoplasmic membrane to the nucleus.

Although previous groups have identified Reg1α in association with many tumor types and to a lesser extent Reg3α has also been described as a tumor marker in primary hepatocellular carcinoma, these findings indicate that Reg plays a direct role in stimulating cancer cells. These findings parallel those of Van Ba in rats, where Megi was found to be over expressed in the hippocampus region of the brain. Studies found that recombinant Reg1α working through Megi resulted in increased neurite outgrowth and this effect was abolished by either inhibiting Reg1α secretion or by downregulating Megi by shRNA. The downregulation of Megi, inactivated the Reg1a activity, with this study finding Megi to be the key receptor for Reg1α in the rat hippocampus. (Van Ba J., et al., Biol Chem. 2011 Dec. 9, www.jbc.org/cgi/doi/10.1074/jbc.M111.260349 Epub ahead of print).

### Example 5

### Design of Inhibitory Antibodies

Studies were undertaken to develop an inhibitory compounds to delay the progression of tumors, which express MEGI and/or Reg1α and Reg3α. For the production of inhibitory antibodies, sequences were evaluated within the N-terminal portion of Megi (amino acids 1-332) (SEQ ID NO: 11), which is the amino acid region of Megi that is not contained in the other members of the of Exostoses family, and thus is hypothesized to be the Reg binding domain.

Consistently, in Enzyme ImmunoAssay studies measuring titers from peptide sequences within SEQ ID NO: 11, resulted in very high polyclonal antibodies being raised to a 20 amino acid Megi sequence of 20 amino acid peptide (SEQ ID NO:4) (amino acids 17-36). The results of the Enzyme ImmunoAssay are summarized in FIG. 10. FIG. 11 demonstrates the standard protocol used for development of polyclonal antibodies to peptide regions within Megi.

Data sets were taken from the bleed after the day 0 and day 21 boosts. The animals were injected with a peptide of SEQ ID NO: 4 and were conjugated to keyhole limpet hemocyanin (KLH). The screening antigen is not conjugated to KLH so that the response solely to the peptide and not to KLH can be identified. The 50% titer is a dilution value where the signal is half-way between the peak and the baseline, so the higher the dilution value (titer), the greater the response to the antigen. The positive control is an internal control that was generated from ovalbumin antibodies in rabbit. At a dilution of 1:759,000, the absorbance fell within a range of 0.45 to 0.9. In the case of the response to SEQ ID NO: 4, there was a high response. The data shown in FIG. 9 from the prebleed sample obtained on Dec. 8, 2010 was <100 (negative) for both rabbits, CD 153 and CD 154. The test bleed taken 31 days after the day 0 and 21 day boosts for CD 153 showed a 50% titer of 36,000 which is an average response, and CD 154 showed a 50% titer of 125,000 which is a high response according to the polyclonal titer reference range shown in FIG. 10. Studies are underway to evaluate the efficacy of the antibody generated with and without the presence of Reg peptide demonstrating that the antibodies raised are inhibitory to the Reg peptide interaction with Megi in pancreatic adenocarcinoma cells.

### Example 6

### Megi Therapeutic and Preventive Vaccines

Based upon the antibody response to SEQ ID NO: 4 within Megi, a therapeutic vaccine development is underway for the treatment of pancreatic cancer and tumors that express Megi. This vaccine may also prove efficacious as a preventive vaccine in populations at risk for pancreatic cancer including patients with diabetes, pancreatitis and family history of pancreatic cancer.

### Example 7

### Screening technique in Serum and Tissue for Megi, Reg1a and Reg3a

To date, there are no biomarkers for pancreatic cancer. This invention finds that Megi, Reg1α and Reg3α are critical to cancer cell growth and expansion. In normal healthy tissue, serum and pancreatic secretions, there is no expression of Megi, Reg1α or Reg3α. Based upon findings in this invention, the presence of Megi, Reg1α and Reg3α is pathological. Presence of Megi, Reg1α and Reg3α in tumor tissue provides new treatment options that are specific with new therapeutics under development to inhibit the action of Reg1α and Reg3α on Megi in order to reduce tumor speed of growth.

When detected in serum, Megi, Reg1α and Reg3α can be specifically used as a tumor maker for earlier diagnosis of cancer and providing the possibility of cure for pancreatic cancers and other occult neoplasms that are only found after there is significant cancer spread. Serum levels of Megi, Reg1α and Reg3α can also be utilized to evaluate the therapeutic efficacy of treatment.

Both plasma and tissue biomarkers for Megi, Reg1α and Reg3α, both for earlier diagnosis in plasma and for decision-making in therapeutic options when Megi, Reg1α or Reg3α are present on tumor cells, are developed utilizing testing including 1) immunohistochemical studies (for example, monoclonal anti-Megi antibody on human cancer cells, anti-human Reg1α antibody on human cancer cells, and anti-human Reg3α antibody on human cancer cells and 2) plasma assay utilizing reverse transcription polymerase chain reaction the exponential amplification via reverse transcription polymerase chain reaction for a highly sensitive evaluation of Megi, Reg3α and Reg1α as a tool for both earlier identification and monitoring treatment status.

These findings show that novel tools for serum screening for Megi, Reg1α and Reg3α for populations at risk, including, for example, those patients with diabetes who have a marked increased risk for both pancreatic and breast cancer, which includes patients with both type 1 and type 2 diabetes who carry a two-fold or greater risk for pancreatic cancer and women with diabetes whose risk is 37% higher than nondiabetic individuals.

All cited references are expressly incorporated herein in their entireties for all purposes. While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

1. A construct comprising at least 7-nucleotides of human, mouse or rat REG gene family cDNA, or the complement thereof, wherein said cDNA is selected from the group consisting of MEGI, REG 1α, REG 1b, REG 3α and REG IV, and wherein the delivery of said construct to a tumor cell results in the inhibition of the expression of said REG gene family cDNA.

2. The construct of claim 1 wherein said construct is selected from the group consisting of an antisense construct and an RNA interference construct.

3. The construct of claim 2, wherein said cDNA is a nucleotide sequence that encodes an amino acid sequence selected from the group consisting of SEQ ID NOs: 1, 5, 6, 7, and 8.

4. The construct of claim 1 wherein said construct comprises at least 15 nucleotides of the complement of human, mouse or rat REG gene family cDNA selected from the group consisting of MEGI, REG 1α, REG 1b, REG 3α and REG IV.

5. A method of treating cancer comprising:
delivering to a tumor cell that expresses a human protein selected from the group consisting of Megi, Reg1α, Reg1β, Reg3α and RegIV the construct of claim 1 in an amount sufficient to reduce the rate of tumor growth in said tumor cell.

6. The method of claim 5, wherein said cancer is a pancreatic cancer.

7. The method of claim 6 wherein said cDNA is a nucleotide sequence that encodes an amino acid sequence selected from the group consisting of SEQ ID NOs: 1, 5, 6, 7, and 8

8. An inhibitor that prevents binding of a rat, mouse, or human Megi protein to a rat, mouse, or human Reg protein selected from the group consisting of Reg1α, Reg1β, Reg3α and RegIV, wherein the delivery of said inhibitor to a tumor cell results in the reduction of resistance to apoptosis by said tumor cell.

9. The inhibitor of claim 8, wherein said inhibitor is selected from the group consisting of an inhibitory antibody and a therapeutic vaccine.

10. The inhibitor of claim 9, wherein said inhibitor is derived from an amino acid sequence selected from the group consisting of SEQ ID Nos: 4-11.

11. A method of treating cancer comprising:
delivering to a tumor cell that expresses a human protein selected from the group consisting of Megi, Reg1α, Reg1β, Reg3α and RegIV the inhibitor of claim 10 in an amount sufficient to reduce the rate of tumor growth in said tumor cell.

12. The method of claim 11, wherein said cancer is a pancreatic cancer.

13. The method of claim 11, wherein said inhibitor is an antibody.

14. The method of claim 13, wherein said antibody is derived from SEQ ID NO: 4.

15. The method of claim 11 wherein said inhibitor is a therapeutic vaccine.

16. A method of treating cancer comprising delivering to tumor cells that express a human protein selected, from the group consisting of Megi, Reg1α, Reg1β, Reg3α and RegIV an antibody which specifically binds to said protein in an amount effective to decrease the rate of growth of said tumor cell.

17. The method of claim 16, wherein said antibody binds to an amino acid sequence comprising SEQ ID NO: 11.

18. The method of claim 17, wherein said cancer is pancreatic cancer.

19. A method of treating and preventing cancer comprising:
Screening a patient for expression of a human protein selected from the group consisting of Megi, Reg1α, Reg1β, Reg3α and RegIV; and
Treating said patient.

20. The method of claim 19, wherein said human protein comprises an amino acid sequence selected from the group consisting of SEQ ID NOs: 1, and 4-11.


SEQUENCE LISTING (13 sequences)

SEQ ID NO: 1 - protein, 919 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPPQVPVKRKYLFTFQGEKIESLRSSLQEARSFEEEMEGDPPADYDDRIIATLKAVQDSKLDQVLVEFTCKNQPKPSLPTEWALCGEREDRLELLKLSTFALIITPGDPRLVISSGCATRLFEALEVGAVPVVLGEQVQLPYQDMLQWNEAALVVPKPRVTEVHFLLRSLSDSDLLAMRRQGRFLWETYFSTADSIFNTVLAMIRTRIQIPAAPIREEAAAEIPHRSGKAAGTDPNMADNGDLDLGPVETEPPYASPRYLRNFTLTVTDFYRSWNCAPGPFHLFPHTPFDPVLPSEAKFLGSGTGFRPIGGGAGGSGKEFQAALGGNVPREQFTVVMLTYEREEVLMNSLERLNGLPYLNKVVVVWNSPKLPSEDLLWPDIGVPIMVVRTEKNSLNNRFLPWNEIETEAILSIDDDAHLRHDEIMFGFRVWREARDRIVGFPGRYHAWDIPHQSWLYNSNYSCELSMVLTGAAFFHKYYAYLYSYVMPQAIRDMVDEYINCEDIAMNFLVSHITRKPPIKVTSRWTFRCPGCPQALSHDDSHFHERHKCINFFVKVYGYMPLLYTQFRVDSVLFKTRLPHDKTKCFKFI

SEQ ID NO: 2 - protein, 23 aa, Homo sapiens
LTWLSFTLFVILVFFPLIAHYYL

SEQ ID NO: 3 - protein, 25 aa, Artificial Sequence
  Description of Artificial Sequence Synthetic peptide
CKKLTWLSFTLFVILFFPLIAHYYL

SEQ ID NO: 4 - protein, 20 aa, Artificial Sequence
  Description of Artificial Sequence Synthetic peptide
CKKSIENAKQDLLQLKNVIS

SEQ ID NO: 5 - protein, 166 aa, Homo sapiens
MAQTSSYFMLISCLMFLSQSQGQEAQTELPQARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFQKWKDVPCEDKFSFVCKFKN

SEQ ID NO: 6 - protein, 166 aa, Homo sapiens
MAQTNSFFMLISSLMFLSLSQGQESQTELPNPRISCPEGTNAYRSYCYYFNEDPETWVDADLYCQNMNSGNLVSVLTQAEGAFVASLIKESSTDDSNVWIGLHDPKKNRRWHWSSGSLVSYKSWDTGSPSSANAGYCASLTSCSGFKKWKDESCEKKFSFVCKFKN

SEQ ID NO: 7 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 8 - protein, 158 aa, Homo sapiens
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 9 - protein, 6 aa, Homo sapiens
IGLHDP

SEQ ID NO: 10 - protein, 14 aa, Artificial Sequence
  Description of Artificial Sequence Synthetic peptide
IGLHDPTQGTEPNG

SEQ ID NO: 11 - protein, 333 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPP

SEQ ID NO: 12 - protein, 5 aa, Artificial Sequence
  Description of Artificial Sequence Synthetic peptide
CKKLT

SEQ ID NO: 13 - protein, 5 aa, Artificial Sequence
  Description of Artificial Sequence Synthetic peptide
CKKSI